(19) United States
(12) Patent Application Publication
(10) Pub. No.: US 2025/0275730 A1
(43) Pub. Date: Sep. 4, 2025

(54) AUTOMATION OF THE BLOOD INPUT FUNCTION COMPUTATION PIPELINE FOR DYNAMIC FDG PET FOR HUMAN BRAIN USING MACHINE LEARNING

(71) Applicant: University of Virginia Patent Foundation, Charlottesville, VA (US)

(72) Inventors: Bijoy Kundu, Charlottesville, VA (US); Gabriel Hyman, Boston, MA (US); Zoraiz Qureshi, Framingham, MA (US); Mark S. Quigg, Charlottesville, VA (US); Nivetha Jayakumar, Charlottesville, VA (US)

(73) Assignee: University of Virginia Patent Foundation, Charlottesville, VA (US)

(21) Appl. No.: 18/854,483

(22) PCT Filed: Apr. 6, 2023
(86) PCT No.: PCT/US2023/017697

§ 371 Date: Oct. 4, 2024

Related U.S. Application Data
(60) Provisional application No. 63/327,970, filed on Apr. 6, 2022.
(60) Provisional application No. 63/329,057, filed on Apr. 8, 2022.

(51) Int. Cl. A61B 6/03; A61B 6/00; A61B 6/46; G06T 7/00; G06T 7/10

(52) U.S. Cl. CPC A61B 6/037; A61B 6/461; A61B 6/5217; G06T 7/0012; G06T 7/10; G06T 2207/10104; G06T 2207/20084; G06T 2207/30016

(57) ABSTRACT

In some examples, method for automatically computing a blood input function for dynamic positron emission tomography (PET) includes obtaining dynamic PET image data sets comprising volumetric radioactive measurement data associated with an administered radioactive tracer present in a target site of a subject over multiple scanning intervals. The method includes utilizing an artificial neural network (ANN) to segment the dynamic PET image data sets displaying one or more blood vessels in the target site. The method includes automatically deriving, using the ANN, a blood input function (I D I F) based on radioactive tracer concentrations measured in one or more segments of the plurality of dynamic PET image data sets. The method includes computing a predictive model-corrected blood input function (MCIF) using time activity curve input associated with the automatically derived IDIF.

### CROSS-REFERENCE TO RELATED APPLICATIONS

This application claims benefit of U.S. Provisional Application Ser. No. 63/327,970, filed on Apr. 6, 2022, and U.S. Provisional Application Ser. No. 63/329,057, filed on Apr. 8, 2022. The disclosure of each of these applications is incorporated herein by reference in its entirety.

### GRANT STATEMENT

This invention was made with government support under Grant No. HL 123627 awarded by the National Institutes of Health. The government has certain rights in the invention.

### BACKGROUND

Positron emission tomography (PET) is a medical imaging technique that allows doctors to see how the body's organs and tissues are working. PET uses a special type of camera to detect positrons, which are emitted from radioactive tracers injected into the body. The tracers travel through the body and collect in areas where there is increased metabolic activity. This information can then be used to create images of the body's organs and tissues.

The blood input function (AIF) is a mathematical model that describes the concentration of a radioactive tracer in arterial plasma as a function of time. The AIF is used to calculate the amount of tracer that has been metabolized by the body, which can then be used to create images of the body's organs and tissues.

The AIF is typically measured by injecting a radioactive tracer into an artery and then using a PET scanner to measure the concentration of the tracer in the blood over time. The AIF can also be estimated using mathematical models based on the PET images.

The AIF is an important tool for PET imaging because it allows doctors to accurately measure the amount of tracer that has been metabolized by the body. This information can then be used to create images of the body's organs and tissues that are more accurate than images that are created without the AIF.

### SUMMARY

In some examples, method for automatically computing a blood input function for dynamic positron emission tomography (PET) includes obtaining a plurality of dynamic PET image data sets comprising volumetric radioactive measurement data associated with an administered radioactive tracer present in a target site of a subject over multiple scanning intervals. The method includes utilizing an artificial neural network (ANN) to segment the plurality of dynamic PET image data sets displaying one or more blood vessels in the target site. The method includes automatically deriving, using the ANN, a blood input function (IDIF) based on radioactive tracer concentrations measured in one or more segments of the plurality of dynamic PET image data sets. The method includes computing a predictive model-corrected blood input function (MCIF) using time activity curve input associated with the automatically derived IDIF.

The computer systems and methods described herein may be implemented in software in combination with hardware and/or firmware. For example, the subject matter described herein may be implemented in software executed by a processor. In one example implementation, the subject matter described herein may be implemented using a non-transitory computer readable medium having stored therein computer executable instructions that when executed by the processor of a computer control the computer to perform steps. Example computer readable media suitable for implementing the subject matter described herein include non-transitory devices, such as disk memory devices, chip memory devices, programmable logic devices, field-programmable gate arrays, and application specific integrated circuits. In addition, a computer readable medium that implements the subject matter described herein may be located on a single device or computer platform or may be distributed across multiple devices or computer platforms.

### BRIEF DESCRIPTION OF DRAWINGS

FIG. 1 is a block diagram of an example system for PET scanning;

FIG. 2 is a block diagram of an example image analyzer;

FIG. 3 is a block diagram of an example 3D UNET architecture for automated segmentation;

FIGS. 4 and 5A-5D illustrate an example implementation of a system for automated prediction of MCIF from segmented IDIF and experimental results;

FIGS. 6A-6E illustrate an example end-to-end pipeline for dynamic FDG PET parametric mapping;

FIG. 7A shows visuals of one example subject with the input 3D reference frame, ground truth, and predicted binary segmentation masks;

FIG. 7B shows segmentation metrics;

FIG. 7C shows a downstream Ki map;

FIG. 8 is a table showing training loss, test loss, average test dataset image wise segmentation metrics; and

FIGS. 9A-9E illustrate an end-to-end pipeline evaluation for an example epileptic patient.

### DETAILED DESCRIPTION

Dynamic fluoro-2-deoxy-D-glucose positron emission tomography (dFDG-PET) is an emerging extension of existing static PET imaging technology. Notably, dFDG-PET allows for the measurement of time dependent information on glucose uptake in the brain. Quantitative analysis of dFDG-PET scans requires the blood input function (IDIF), which describes the level of radiotracer within the blood that is available for tissues to use, as a means of calibrating regional dynamic glucose changes. While in animal models, the IDIF is captured through arterial sampling, a non-invasive approach is preferred for humans to avoid the associated risks (e.g., arterial occlusion and infection) with arterial sampling.

This document describes a fully automatic pipeline that can facilitate the calculation of an accurate IDIF thereby enabling accurate prediction of MCIF which may result in higher resolution computed parametric PET maps. Currently, the derivation of this IDIF for brain dFDG-PET is manual, making it an imprecise and time-consuming process. Manual segmentation of the carotid arteries, which supply the brain with oxygenated blood, is the current gold standard target for deriving the IDIF. This manual annotation involves using a software that traces the boundaries of the left and right carotid arteries within the scan data. The IDIF is calculated from measuring the radiotracer concentration across time, also known as the time activity curve (TAC), within these segments of the image. We developed a supervised artificial neural network (ANN) trained on manual annotations to segment the carotid arteries in brain dFDG-PET, allowing for the automatic derivation of the IDIF, and, ultimately, subsequent brain wide parametric mapping of cerebral metabolism.

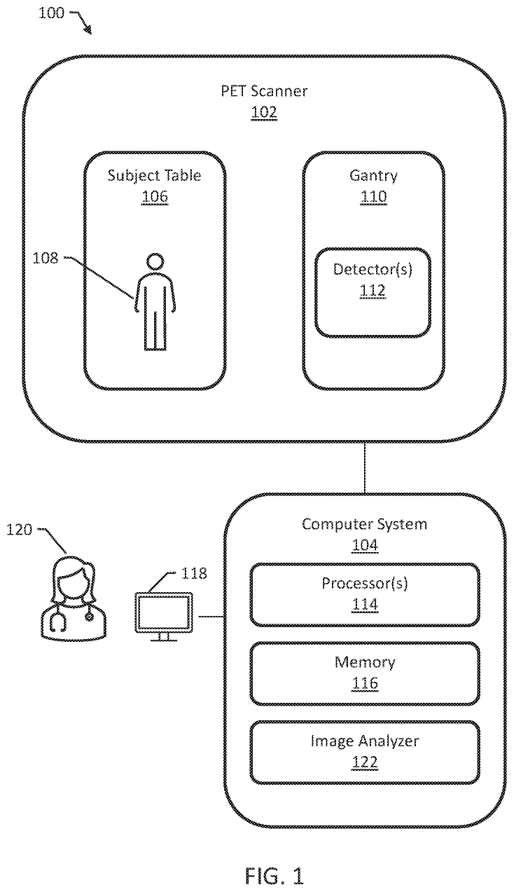

FIG. 1 is a block diagram of an example system 100 for PET scanning. The system 100 includes a PET scanner 102 and a computer system 104 programmed for image analysis.

The PET scanner 102 includes a subject table 106 for a subject 108 to lie on during the PET scan. The PET scanner 102 also includes a gantry 110 and one or more detectors 112. The gantry 110 is a frame that holds the detectors 112. The gantry 110 rotates around the subject table 106, and the detectors 112 rotate with the gantry 110. The detectors 112 are sensitive to the positrons emitted from the radioactive tracers. For example, the detectors 112 can be made of a scintillator material, which is a material that emits light when it is struck by radiation. The light is then converted into an electrical signal by a photomultiplier tube (PMT). The PMT amplifies the electrical signal and sends it to the computer system for image analysis.

The computer system 104 for image analysis is used to process the images from the PET scanner 102. The computer system 104 includes one or more processors 114, memory 116 storing instructions for the processors 114, and a display device 118 for displaying PET images to a user 120. The user 120 can be, e.g., a medical professional or a researcher. The computer system allows the user 120 to create high-quality images of the body's organs and tissues. The images can be used, e.g., for research, and to diagnose diseases, to plan treatments, and to monitor the progress of treatment.

The computer system 104 includes an image analyzer 122 configured for producing and processing PET images. The images are first reconstructed, which means that they are converted from the raw data collected by the detectors 112 into images that can be viewed by the user 120. The images can then be processed to remove noise and to improve the contrast. The images can also be used to create 3D images of the body's organs and tissues.

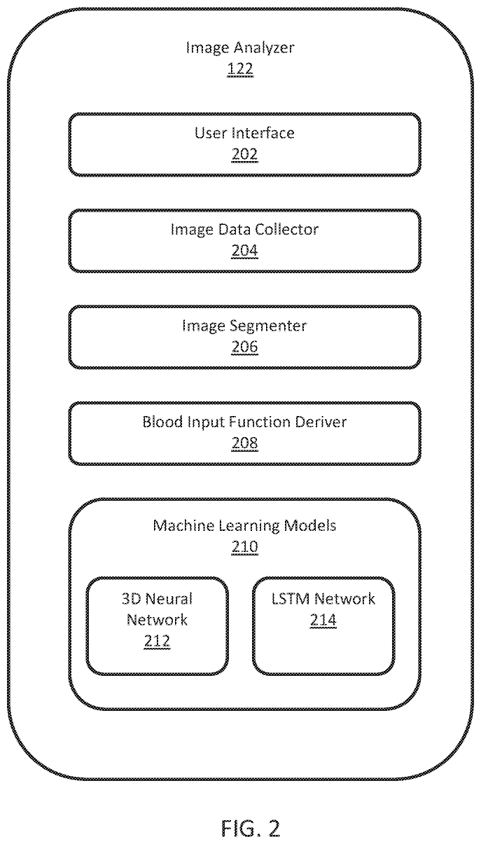

FIG. 2 is a block diagram of an example image analyzer 122. The image analyzer 122 includes a user interface 202, and image data collector 204, an image segmenter 206, and a blood input function deriver 208. The image analyzer 122 can include one or more machine learning models 210, e.g., for image segmentation and blood input function derivation. As shown in the example of FIG. 2, the image analyzer 122 includes a 3D neural network 212 and a long short term memory (LSTM) network 214.

The user interface 202 can be a graphical user interface (GUI) that includes a series of windows and menus that allow users to interact with the system. The user interface 202 can be configured for any appropriate tasks, e.g., configuring and operating the computer system, viewing and analyzing PET images, saving and printing PET images, sharing PET images with other users, generating reports on PET images, and managing PET data.

The image data collector 204 is configured for collecting dynamic PET image data sets comprising volumetric radioactive measurement data associated with an administered radioactive tracer present in a target site of a subject over multiple scanning intervals. The image data collector 204 can also be configured for any appropriate type of image pre-processing.

The image segmenter 206 is configured for utilizing an artificial neural network (ANN) to segment the plurality of dynamic PET image data sets displaying one or more blood vessels in the target site. The image segmenter 206 can use the 3D neural network 212, e.g., an end-to-end 3D convolutional neural network, for segmentation.

The blood input function deriver 208 is configured for automatically deriving, using the ANN, a blood input function (IDIF) based on radioactive tracer concentrations measured in one or more segments of the plurality of dynamic PET image data sets. The blood input function deriver 208 is configured for computing a predictive model-corrected blood input function (MCIF) using time activity curve input associated with the automatically derived IDIF. The blood input function deriver 208 can use the LSTM network 214, e.g., which can be trained over a time-distributed dense layer to predict the MCIF as output directly from the IDIF.

LSTM is a type of recurrent neural network architecture that is commonly used for processing sequential data such as time series, speech, and text. The key feature of LSTM is its ability to capture long-term dependencies in the input sequence. Traditional recurrent neural networks suffer from the vanishing gradient problem, where the gradients become too small as they are backpropagated through time, leading to difficulties in training the network to capture long-term dependencies. LSTMs solve this problem by introducing memory cells, which can store information over long periods of time and selectively forget or add new information to the cell state based on the input and past history.

LSTM networks typically have three gates—the input gate, output gate, and forget gate—which control the flow of information into and out of the memory cell. The input gate determines which information from the input sequence should be stored in the cell state, while the forget gate decides which information should be discarded. The output gate controls the output of the LSTM at each time step by selectively revealing or hiding information from the memory cell.

FIG. 3 is a block diagram of an example 3D UNET architecture for automated segmentation, e.g., of the internal carotid arteries from dFDG PET images of the human brain to derive IDIF. The 3D UNET can be trained on training images with manual annotations to segment the carotid arteries of the brain or other appropriate anatomical structures, allowing for automatic derivation of the IDIF, and ultimately subsequent brain wide parametric mapping of cerebral metabolism.

A 3D-UNET is a type of neural network architecture that can be used in medical image analysis tasks, such as segmentation of organs or lesions from medical images. It is an extension of the original UNET architecture that was developed for 2D image segmentation tasks.

The 3D-UNET architecture typically uses a fully convolutional neural network with an encoding and decoding path. The encoding path is composed of several convolutional layers that extract high-level features from the input 3D image. The decoding path, which is symmetric to the encoding path, uses a series of upsampling and concatenation operations to generate a segmentation map that has the same size as the input image.

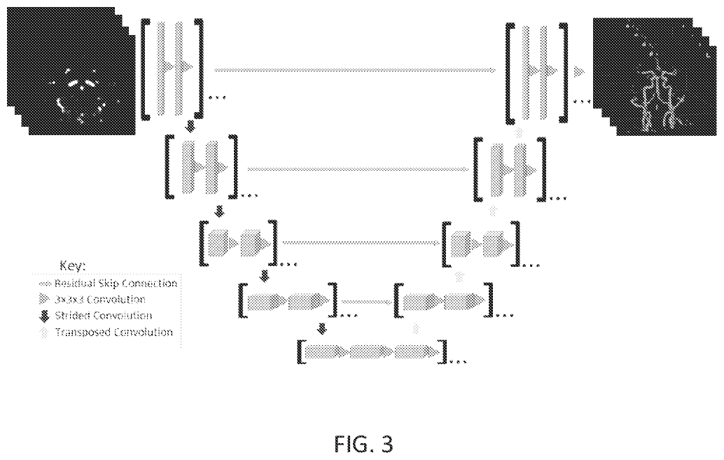

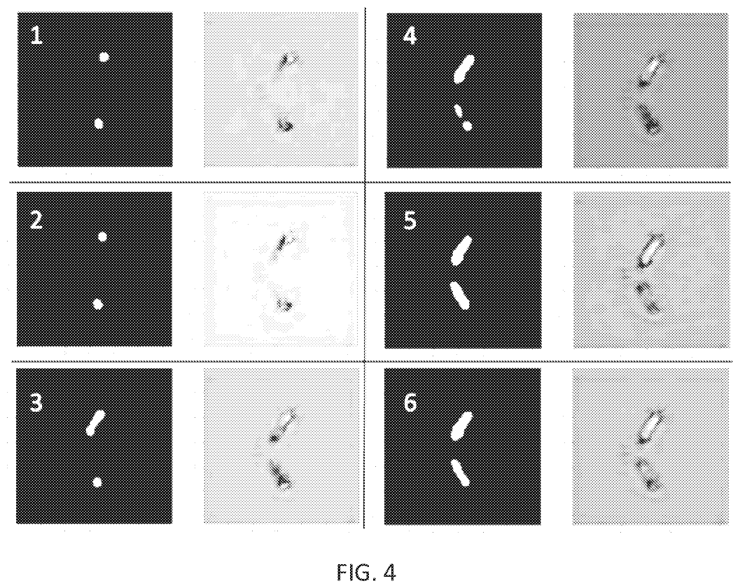

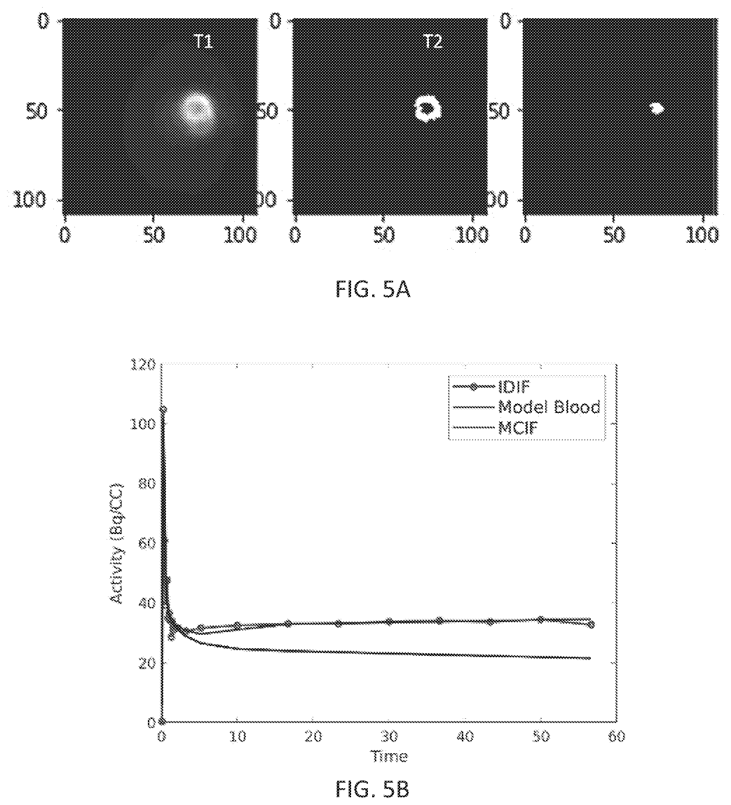

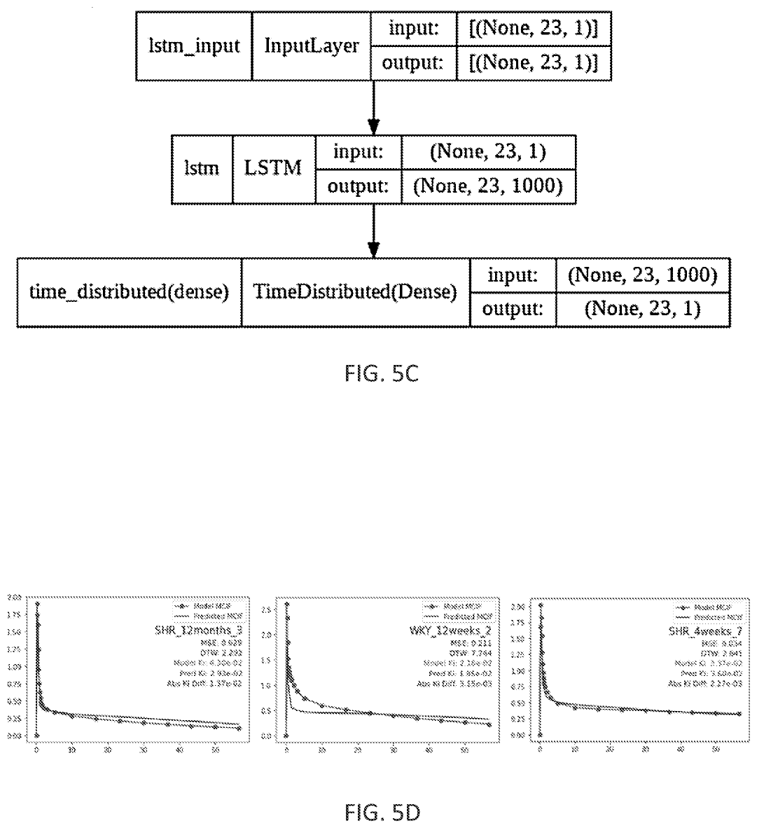

FIGS. 4 and 5A-5D illustrate an example implementation of a system for automated prediction of MCIF from segmented IDIF and experimental results. Preliminary computations were performed on 36 dynamic FDG brain PET data sets obtained at the University of Virginia (25:6:5 (70:15:15%) split-train:val:test) utilizing baseline 3D-UNET in Google Colab with a binary cross entropy loss function, ˜130 k model parameters and 40 epochs (˜90 minutes).

FIG. 4 shows a side-by-side comparison of manually and network generated annotation. Six slices of both manually (left) and network-generated (right) annotations are shown side-by-side in ascending order.

A major drawback of model based MCIF computations is the manual determination of upper, lower, and initial guess values of 15 model parameters and prior knowledge of the recovery coefficients based on structural imaging. To bypass this manual step, we will automate it as a time-series regression task using a supervised deep learning approach using a LSTM (long-short-term memory) network architecture which belongs to the class of recurrent neural networks. For every dataset, we have obtained segmentations, their corresponding IDIFs and computed MCIFs using the 15-parameter model manually. The next step is to train an LSTM network over a time-distributed dense layer to accurately predict the MCIF as output directly from an input IDIF only, without depending on the previous model to automatically perform the correction for the two contamination effects present in the input IDIF. For example, the disclosed subject matter may include an ANN that comprises an end-to-end three-dimensional (3D) convolutional neural network for segmentation and a LSTM network architecture that is trained over a time-distributed dense layer to predict the MCIF as output directly from the IDIF.

In some embodiments, a loss function such as the mean-squared error (MSE) or root mean-squared-error (RMSE) can possibly be used for this regression, with gradient descent optimizers such as Adam. The metrics for evaluation of the output predictions will include time-series similarity distance metrics such as MSE itself as well as the dynamic-time warping (DTW) distance. Another domain-specific metric we can introduce is the parametric downstream Ki associated to a blood input function where Ki represents the rate of FDG tracer uptake from the blood to the brain tissue. A Ki value can be computed for the predicted MCIF by the model on a test sample and compared to that of the model ground truth MCIF that was manually obtained, and the absolute difference between the predicted and model Ki may serve as a metric possibly better quantify how good the prediction is, where a lower difference would signify a more accurate prediction.

FIGS. 5A-5D illustrate an example application to rodent dynamic cardiac PET data. FIG. 5A shows semi-automated segmentation results for a sample axial slice across a rodent dynamic PET dataset. Firstly, the ring-like myocardial tissue structure segmented for the given slice, using a manual threshold estimate T1 and area-based rules for filtering among disjoint regions. Finally, the internal Left Ventricular blood pool segmented from more complete ring-like myocardial structures formed using a slightly lower threshold T2.

FIG. 5B shows a sample model-corrected blood input function (MCIF) computed using myocardial and LV time-activity curves (the latter also defined as the image-derived input function or IDIF) obtained from the prior segmentation approach as input. Our 3-compartment 15-parameter model corrects for any partial volume averaging and spillover contamination effects present in the IDIF. FIG. 5C shows the model architecture used to predict MCIFs from IDIFs—a single layer of 1000 LSTM cells, then time-distributed over a dense layer to regress the predicted MCIF (23-frame time-series) as output. FIG. 5D shows sample model predictions over different rodent age groups from the repeated random subsampling cross-validation approach with an 70:15:15% train:test:validation split. The means and standard deviations of the various evaluation metrics are shown, including Mean-Squared Error (MSE), Dynamic-Time Warping (DTW), model Ki, predicted Ki and the absolute difference between them.

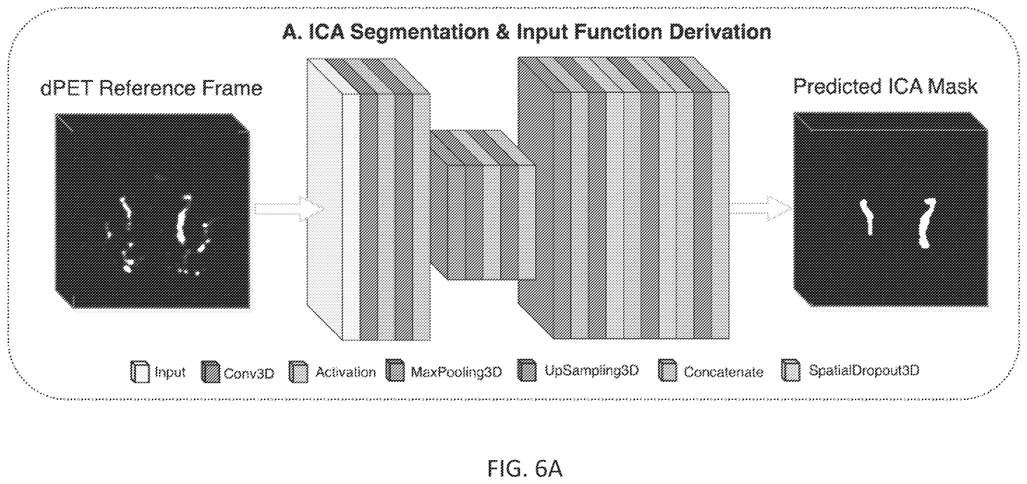

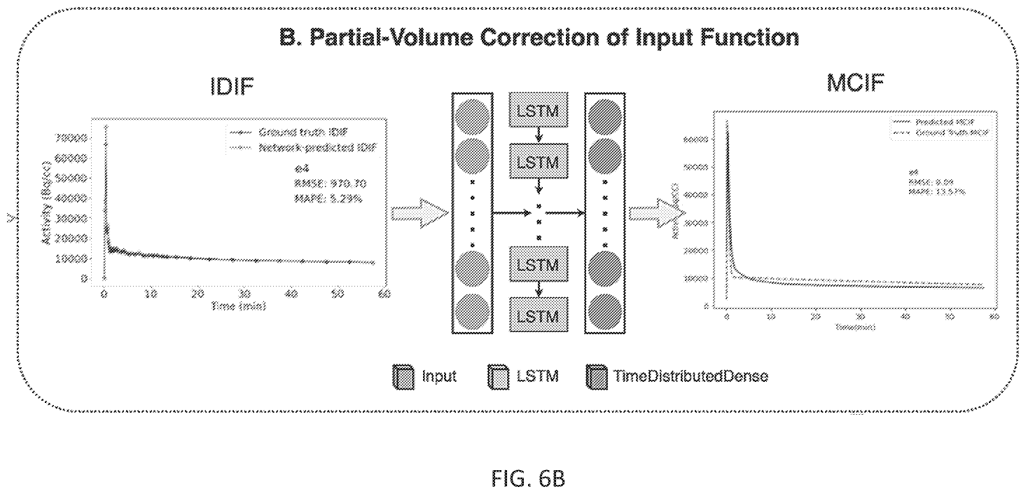

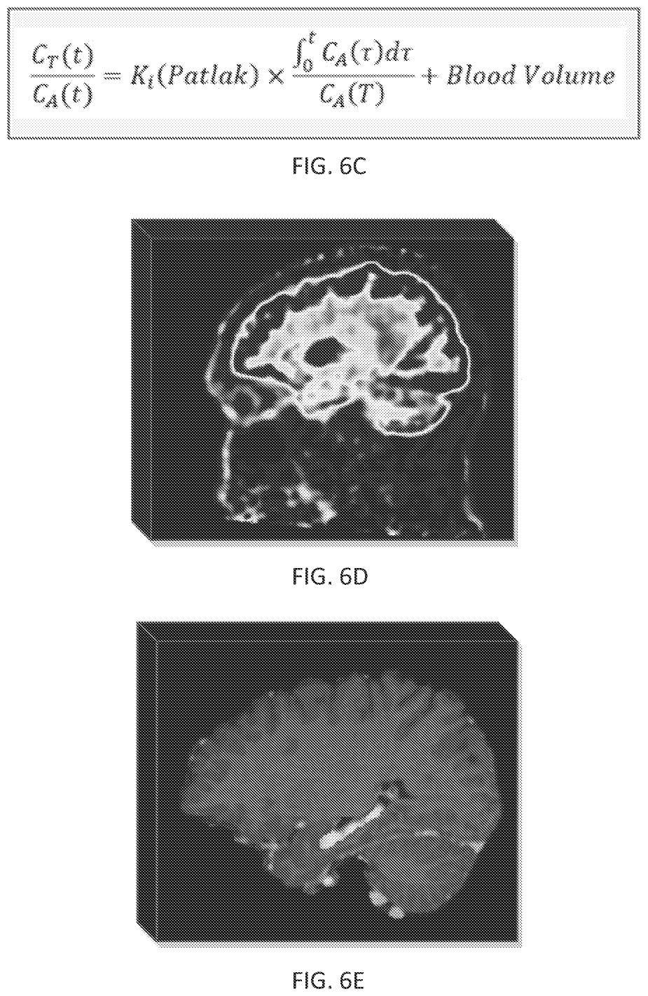

FIGS. 6A-6E, 7A-70, 8, and 9A-9E are directed to a deep learning pipeline for parametric FDG brain PET mapping in localization of focal epilepsy.

Arterial blood sampling can be used for deriving blood input for parametric mapping of dynamic brain PET. Model based computations from the internal carotid arteries (ICA) with partial volume (PV) corrections obviate the need for arterial sampling. This document describes an end-to-end 3D U-Net and Long Short Term Memory (LSTM) deep learning pipeline that can facilitate automatic segmentation of ICA (ICA-net) and derivation of model corrected blood input function (MCIF-net) with PV corrections, respectively, for automatic identification of seizure foci for human dynamic FDG brain PET.

To illustrate the pipeline, a study was performed using an example system. Dynamic FDG PET (dFDG-PET) of the brain was performed for 60 minutes on 40 subjects using a PET CT scanner, preceded by non-contrast T1-weighted MPRAGE MRI scans for motion correction and co-registration. A reference frame containing the ICA was programmatically selected from dynamic 4D PET data through intensity analysis, followed by semi-automatic annotation of the ICA using 3DSlicer. For automated IDIF derivation, a 3D U-Net architecture was trained with the reference frame as the input volume and the binary 3D segmentation mask having the model's prediction of the ICA for the dataset as output. The UNET was designed with a depth of 2, 8 base convolutional filters in the first layer and 3D spatial dropout layers of 0.5. Training was performed over the 40 dFDG-PET datasets using 5-fold cross-validation using the Dice loss function, with a batch size of 1 and Adam optimizer (learning rate=0.001) for 120 epochs per fold using Rivanna high performance computing. A 4-fold data augmentation was applied on the training set for each fold based on rotation, elastic deformation and horizontal flips during training. Downstream IDIFs were computed from the predicted ICA segmentations (ICA-net). An LSTM with 30 units, Tanh activation and Adam optimizer (learning rate=0.0001) was developed to map IDIF to MCIF. Model MCIF was optimized and computed using a multi-parameter 3-compartment model with spill-over and PV corrections. The model was trained for 5000 epochs using 5-fold cross-validation to evaluate the performance of the LSTM. The metrics used for assessing agreement were the loss function root mean-squared error (RMSE) and mean absolute error (MAE). Model MCIF derived downstream whole brain Ki were compared with predicted MCIF derived Ki computed using a graphical Patlak model and the absolute difference in Ki was computed. Z-scores from downstream predicted Ki's were computed normalizing for the whole brain (18 super regions/side) mean and standard deviation (SD) for a patient with epilepsy with known surgical ground truth. A cut-off of z score<−1.66 SD was utilized to identify hypometabolic regions.

### RESULTS

ICA-net: For generated test set segmentations, average evaluation metrics across the 5 folds show a Dice Similarity Coefficient or F1 score of 0.808+0.094, Intersection over Union (IoU) of 0.691±0.118, 95% Robust Hausdorff Distance (RHD 95) of 8.753±6.083. MCIF-net: The average normalized RMSE across the 5 folds were 0.3010±0.0127 and the MAPE was 24.157±3.52%. The average downstream absolute voxel-wise Ki difference was 0.0009+0.000089 min-1. Z-score results: The downstream predicted z-score computations with a cut-off of −1.66 SD resulted in identification of right temporal regions for the epileptic patient as hypometabolic (z score of −2.375 for right hippocampus) and an average z-score RMSE of 0.02445 against the ground truth. The patient underwent laser interstitial thermal therapy of the right hippocampus and was seizure free for 30 months.

### CONCLUSION

This work suggests that an end-to-end 3D U-Net and LSTM deep learning pipeline was effectively able to learn the underlying distribution of the target structure of the ICA and could precisely facilitate the automatic computation of the MCIF with PV corrections enabling downstream prediction of seizure foci for dFDG-PET of the human brain.

FIGS. 6A-6E illustrate an example end-to-end pipeline for dynamic FDG PET parametric mapping. After motion correction and coregistration of the dynamic PET scan to MRI, a trained 3D-UNet model is used to predict the ICA segmentation mask from a selected dynamic PET reference frame.

FIG. 6A shows the selected dynamic PET reference frame convolved over the raw 4D PET scan to compute the IDIF. To correct the resultant IDIF for partial-volume correction, a trained sequence-to-sequence LSTM maps the IDIF to an MCIF. FIG. 6B illustrates a graphical Patlak model that takes the MCIF and dynamic PET scan to compute a voxel-wise rate of uptake Ki map. FIG. 6D shows the map. Additionally, for evaluation, regional Z-score masks for the Ki are computed for 18 super-regions of the brain normalized to the whole brain for an epileptic patient with known ground truth. Regions with Z-score<−1.66 standard deviations below the mean are considered hypometabolic to identify seizure foci. FIG. 6E shows a regional voxel-wise Z-score mask with identified seizure foci.

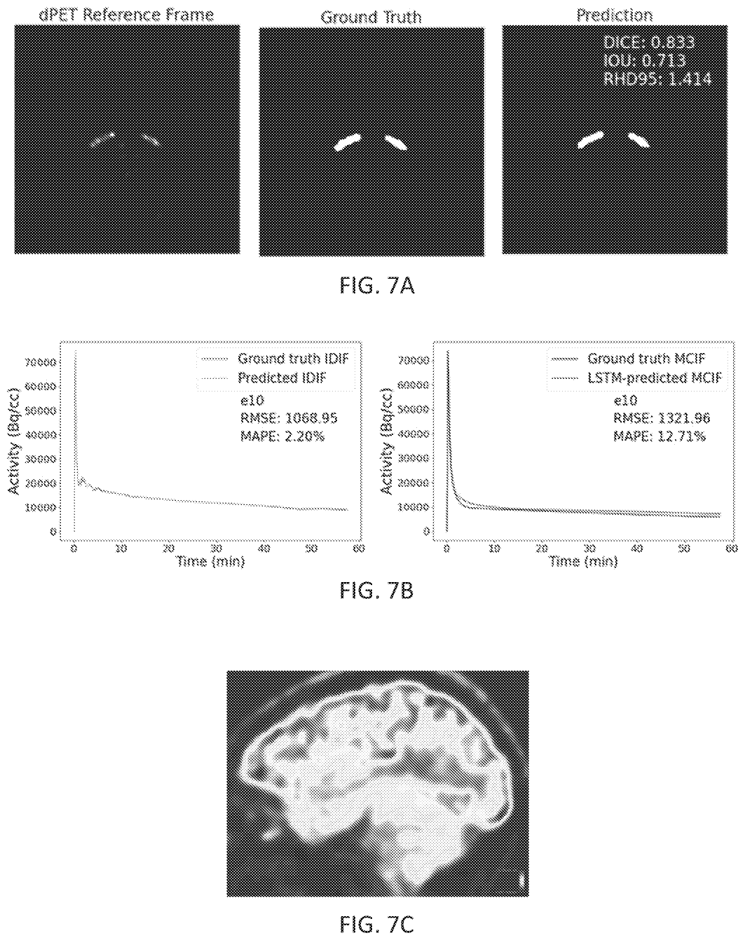

FIG. 7A shows visuals of one example subject with the input 3D reference frame, ground truth, and predicted binary segmentation masks. FIG. 7B shows segmentation metrics (IoU, Dice, 95% Robust Hausdorff Distance) for the ICA-net, downstream IDIF, predicted MCIF, evaluation metrics (RMSE, MAPE) for the MCIF-net. FIG. 7C shows a downstream Ki map.

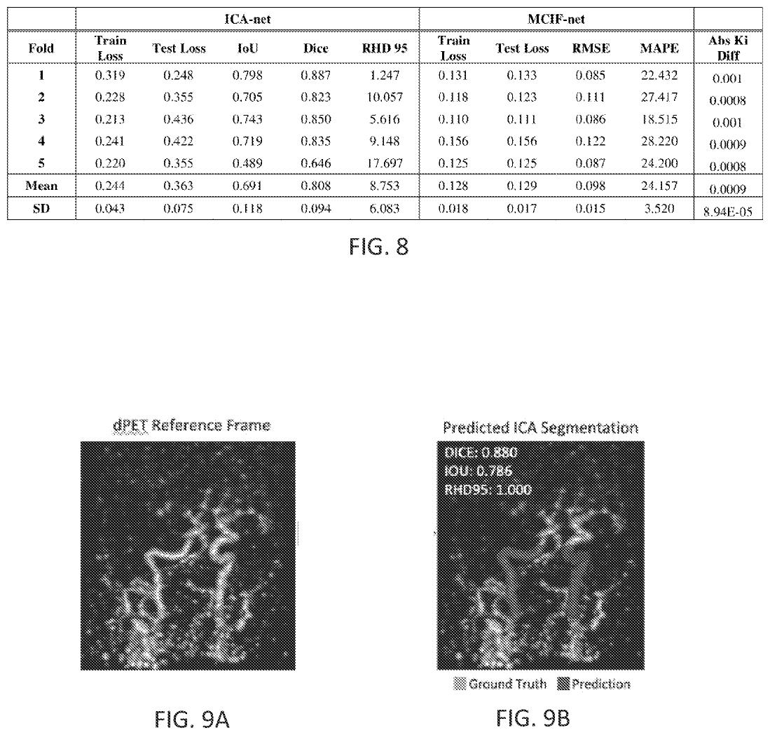

FIG. 8 is a table showing training loss, test loss, average test dataset image wise segmentation metrics (IoU, Dice, 95% Robust Hasudorff Distance) for the ICA-net and MCIF train loss, test loss and evaluation metrics (RMSE, MAPE) for the MCIF-net and absolute different (min⁻¹) for the generated Ki maps, for each fold of the 5-fold cross-validation training across all 40 datasets.

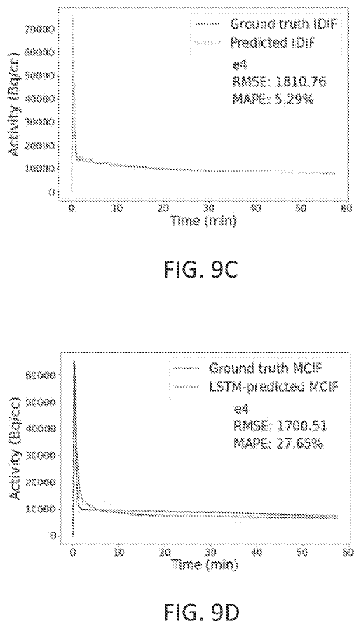

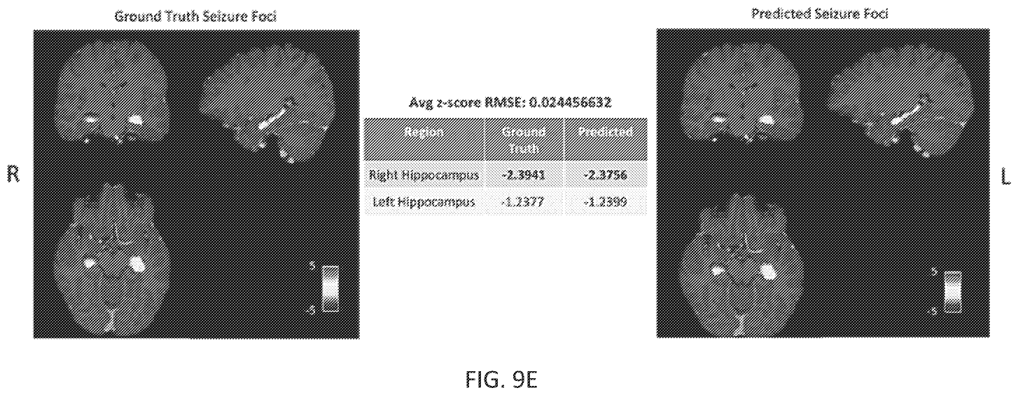

FIGS. 9A-9E illustrate an end-to-end pipeline evaluation for an example epileptic patient, visualizing the raw dFDG-PET reference frame, predicted ICA segmentation from the ICA-net, downstream IDIF, predicted MCIF from the MCIF-net and Z-scores for seizure foci identification (right hippocampus). FIG. 9A shows a dPET reference frame, and FIG. 9B shows a predicted ICA segmentation compared to the ground truth. FIGS. 9C and 9D are graphs illustrating predicted IDIF and LSTM-predicted MCIF compared to ground truth. FIG. 9E shows images for ground truth seizure foci and predicted seizure foci.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for automatically computing a blood input function for dynamic positron emission tomography (PET), the method comprising:
obtaining a plurality of dynamic PET image data sets comprising volumetric radioactive measurement data associated with an administered radioactive tracer present in a target site of a subject over multiple scanning intervals;
utilizing an artificial neural network (ANN) to segment the plurality of dynamic PET image data sets displaying one or more blood vessels in the target site;
automatically deriving, using the ANN, a blood input function (IDIF) based on radioactive tracer concentrations measured in one or more segments of the plurality of dynamic PET image data sets; and
computing a predictive model-corrected blood input function (MCIF) using time activity curve input associated with the automatically derived IDIF.

2. The method of claim 1 wherein the MCIF is used to generate objective parametric PET maps of the target site.

3. The method of claim 1 wherein deriving the IDIF includes continuously collecting the plurality of volumetric radioactive measurements at the multiple scanning intervals over a predefined time period.

4. The method of claim 1 wherein prior to the obtaining step, a subject is injected with the radioactive tracer.

5. The method of claim 1 wherein the dynamic PET includes dynamic fluoro-2-deoxy-D-glucose (dFDG)-PET.

6. The method of claim 1 wherein the one or more blood vessels includes one or more carotid arteries.

7. The method of claim 1 wherein the target site is a human brain.

8. The method of claim 1 wherein the ANN includes an end-to-end 3D convolutional neural network for segmentation and a long-short-term memory (LSTM) network architecture that is trained over a time-distributed dense layer to predict the MCIF as output directly from the IDIF.

9. The method of claim 1 comprising automatically identifying one or more seizure foci for human dynamic FDG brain PET.

10. A system for performing dynamic positron emission tomography (PET), the system comprising:
a PET scanner configured for configured for collecting a plurality of dynamic PET image data sets comprising volumetric radioactive measurement data associated with an administered radioactive tracer present in a target site of a subject over multiple scanning intervals;
a computer system comprising:
at least one processor;
a memory element; and
an image analyzer stored in the memory element and when executed by the at least one processor is configured for:
obtaining the plurality of dynamic PET image data sets from the PET scanner;
utilizing an artificial neural network (ANN) to segment the plurality of dynamic PET image data sets displaying one or more blood vessels in the target site;
automatically deriving, using the ANN, a blood input function (IDIF) based on radioactive tracer concentrations measured in one or more segments of the plurality of dynamic PET image data sets; and
computing a predictive model-corrected blood input function (MCIF) using time activity curve input associated with the automatically derived IDIF.

11. The system of claim 10 wherein the MCIF is used to generate objective parametric PET maps of the target site.

12. The system of claim 10 wherein deriving the IDIF includes continuously collecting a plurality of volumetric radioactive measurements at the multiple scanning intervals over a predefined time period.

13. The system of claim 10 wherein prior to the obtaining step, a subject is injected with the radioactive tracer.

14. The system of claim 10 wherein the dynamic PET includes dynamic fluoro-2-deoxy-D-glucose (dFDG)-PET.

15. The system of claim 10 wherein the one or more blood vessels includes one or more carotid arteries.

16. The system of claim 10 wherein the target site is a human brain.

17. The system of claim 10 wherein the ANN includes wherein the ANN includes an end-to-end 3D convolutional neural network for segmentation and a long-short-term (LSTM) network architecture that is trained over a time-distributed dense layer to predict the MCIF as output directly from the IDIF only.

18. The system of claim 10, wherein the image analyzer is configured for automatically identifying one or more seizure foci for human dynamic FDG brain PET.

19. One or more non-transitory computer readable media having stored thereon executable instructions that when executed by a processor of a computer cause the computer to perform steps comprising:
obtaining a plurality of dynamic PET image data sets comprising volumetric radioactive measurement data associated with an administered radioactive tracer present in a target site of a subject over multiple scanning intervals;
utilizing an artificial neural network (ANN) to segment the plurality of dynamic PET image data sets displaying one or more blood vessels in the target site;
automatically deriving, using the ANN, a blood input function (IDIF) based on radioactive tracer concentrations measured in one or more segments of the plurality of dynamic PET image data sets; and
computing a predictive model-corrected blood input function (MCIF) using time activity curve input associated with the automatically derived IDIF.
